(19) United States
(12) Patent Application Publication
(10) Pub. No.: US 2011/0282222 A1
(43) Pub. Date: Nov. 17, 2011

(54) COILING BLOOD PRESSURE CUFF

(75) Inventors: Chao-Man TSENG, Taipei (TW); Lien-Sheng Cehn, Tucheng City (TW); Yung-Ming Tsai, Taipei City (TW)

(73) Assignee: K-Jump Health Co., Ltd.

(21) Appl. No.: 12/778,358

(22) Filed: May 12, 2010

(51) Int. Cl. A61B 5/022

(52) U.S. Cl. 600/499

(57) ABSTRACT

A coiling blood pressure cuff for winding and compressing a testing position of a human body includes a strip-shaped body, an inflatable bladder, and a flexible elastic plate. The inflatable bladder is installed to the strip-shaped body, and the flexible elastic plate is attached onto the strip-shaped body and along the lengthwise direction of strip-shaped body. If an external force is exerted onto the flexible elastic plate to curl and deform the flexible elastic plate, the flexible elastic plate will be curled and contracted elastically and automatically to drive the strip-shaped body to be wound and positioned at the testing position, so as to provide an easy operation and a quick positioning.

### BACKGROUND OF THE INVENTION

1. Field of the Invention

The present invention generally relates to blood pressure measuring devices, in particular to a coiling blood pressure cuff.

2. Description of Prior Art

Blood pressure is produced at walls of blood vessels when blood is circulated in a human body, and the pressure produced by a heart when the heart is contracted to deliver blood to all part of the body through arteries is called a systolic blood pressure (or a maximum blood pressure) and the pressure produced by the heart when the heart is relaxed to allow blood to return to the heart through veins is called a diastolic blood pressure (or a minimum blood pressure). Blood pressures are measured in the unit of millimeters of mercury (mmHg). According to standards of the World Health Organization, a blood pressure is defined as an abnormal blood pressure if the systolic blood pressure is over 160 mmHg and the diastolic blood pressure is over 95 mmHg, or the systolic blood pressure is below 100 mmHg and the diastolic blood pressure is below 60 mmHg. Abnormal blood pressure is a warning signal of many fatal chronic diseases such as stroke, heart disease, uremia, diabetes and hypertension, etc. Therefore, we can know many hidden diseases of a human body by measuring blood pressures. In general, a sphygmomanometer is usually used for monitoring the condition of the operation between the heart and blood vessels to learn about the health condition of a human body, and providing a positive way of predicting an occurrence of a chronic disease. If the blood pressures can be monitored, observed and recorded in a long term, the record of blood pressures serves as important reference data for personal medical checkups and medications.

A general sphygmomanometer includes an inflatable arm band and a pressure sensing device installed onto the inflatable arm band, and a latch ring is sewed at an end of the inflatable arm band, and a VELCRO® tape is attached on both sides of the inflatable arm band separately. When the sphygmomanometer is used, the inflatable arm band is wound around an examinee's arm, and another end of the inflatable arm band is passed through the latch ring and folded back, and the VELCRO® tapes are used for positioning and binding, and then the inflatable arm band is inflated to compress the examinee's arm. Now, the pressure sensing device is used to measure blood pressures. However, the inflatable arm band will compress the arm and the latch ring will be brought into an abutting contact with the examinee arm after the inflatable arm band is inflated. Users may feel uncomfortable or even painful due to the pressure from the latch ring. Furthermore, it is not easy for the examinee to wind the inflatable arm band around the examinee's arm before taking a blood pressure measurement.

In view of the shortcomings of the conventional sphygmomanometer, the inventor of the present invention based on years of experience in the related industry to conduct extensive researches and experiments, and finally developed a feasible and effective coiling blood pressure cuff in accordance with the present invention to overcome the shortcomings of the prior art.

### SUMMARY OF THE INVENTION

Therefore, it is a primary objective of the present invention to provide a coiling blood pressure cuff that can be deformed flexibly and positioned at a testing position of a human body, so as to provide a convenient operation and a quick positioning effect.

To achieve the foregoing objectives, the present invention discloses a coiling blood pressure cuff, comprising: a strip-shaped body; an inflatable bladder, installed at the strip-shaped body; and a flexible elastic plate, attached onto the strip-shaped body and along the lengthwise direction of the strip-shaped body, such that if an external force is exerted onto the flexible elastic plate to curl and deform the flexible elastic plate, the flexible elastic plate will be curled and contracted elastically and automatically to drive the strip-shaped body to be wound and positioned at the testing position.

The advantages of the present invention are described as follows. When an external force is applied to the flexible elastic plate which is in a rigid straight status at the beginning to bend the flexible elastic plate, the flexible elastic plate will be curled, deformed, and contracted to drive the strip-shaped body to be wound and positioned at the testing position. The invention provides an easy and convenient operation and a quick positioning effect. In addition, the structure of the invention has a very simple structure and a low manufacturing cost, such that the product competitiveness can be improved. The invention further prevents users from being abutted and pressed by a latch ring of the conventional sphygmomanometer, such that the users will not feel uncomfortable due to any sharp pain caused by the latch ring.

### BRIEF DESCRIPTION OF DRAWING

The technical characteristics and contents of the present invention will become apparent with the following detailed description accompanied with related drawings.

FIG. 1 is an exploded view of the present invention;

FIG. 2 is a perspective view of the present invention;

FIG. 3 is a cross-sectional view of Line 3-3 of FIG. 2;

FIG. 4 is a schematic view of an application of the present invention;

FIG. 5 is a cross-sectional view of the present invention automatically curled at a testing position;

FIG. 6 is a cross-sectional view of the present invention wound and fixed at a testing position;

FIG. 7 is an exploded view of another preferred embodiment of the present invention; and

FIG. 8 is a cross-sectional view of another preferred embodiment of the present invention.

### DETAILED DESCRIPTION OF THE INVENTION

The technical characteristics and contents of the present invention will become apparent with the following detailed description accompanied with related drawings.

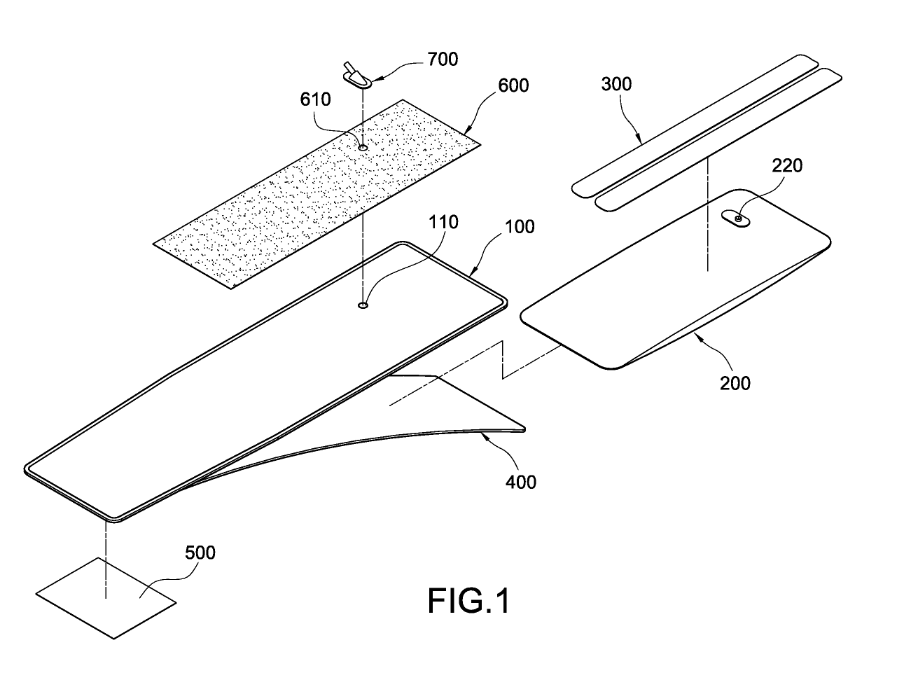

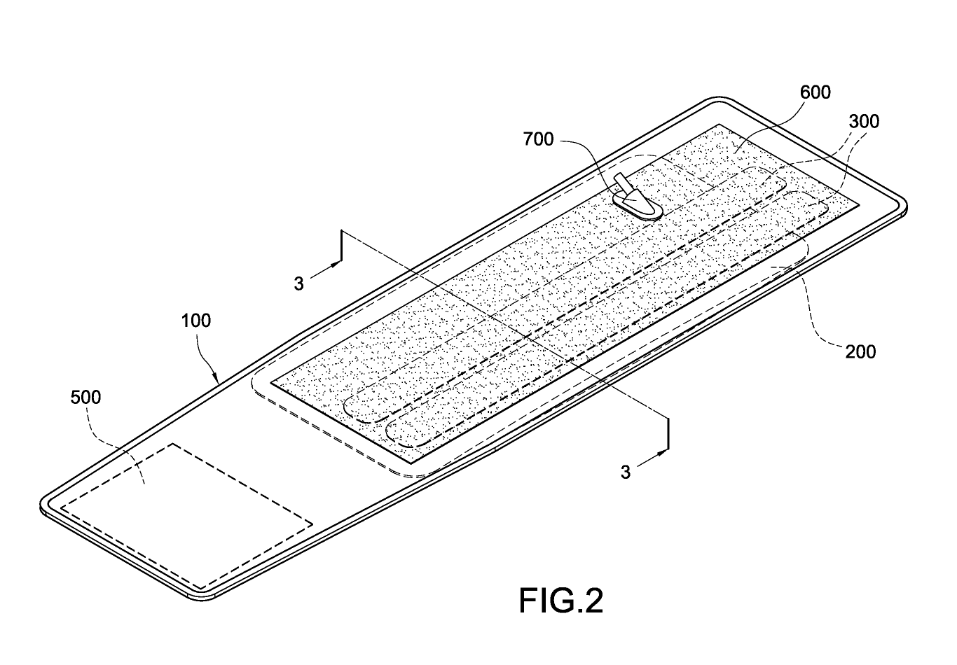

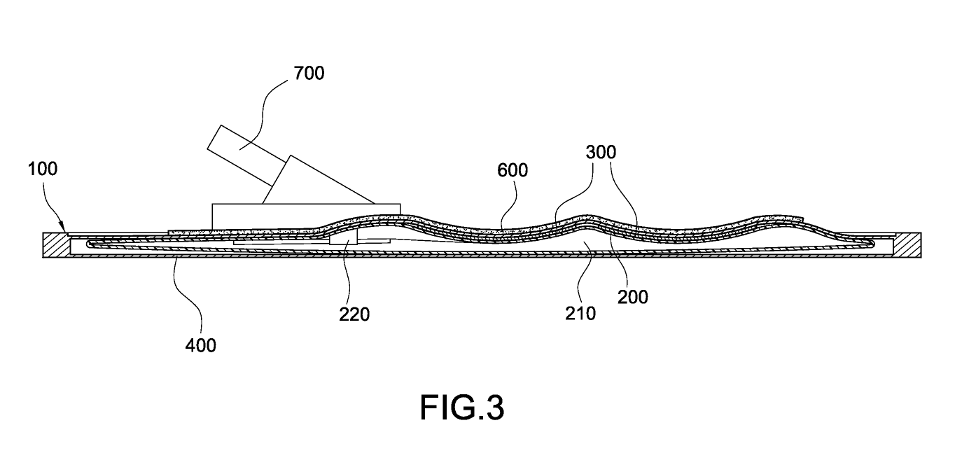

With reference to FIGS. 1 to 3 for a coiling blood pressure cuff of the present invention, the coiling blood pressure cuff comprises a strip-shaped body 100, an inflatable bladder 200, a flexible elastic plate 300, a bottom lining 400, a male adhesive layer 500, a female adhesive layer 600, and a vent connector 700.

The strip-shaped body 100 is made of a soft material having a better tension to assure its durability, but the invention is not limited to such material only. The strip-shaped body 100 is substantially in a rectangular shape, but the invention is not limited to such shape only. In addition, the strip-shaped body 100 includes an interconnecting hole 110 formed thereon.

The inflatable bladder 200 is installed on a side of the strip-shaped body 100 and can be sewed or fixed directly onto the strip-shaped body 100, and the inflatable bladder 200 contains an air chamber 210, and the inflatable bladder 200 includes an inflation hole 220 interconnected to the air chamber 210, and the inflation hole 220 is interconnected to the interconnecting hole 110.

The flexible elastic plate 300 is attached onto the strip-shaped body 100 and along the lengthwise direction of the strip-shaped body 100. The flexible elastic plate 300 and the inflatable bladder 200 are installed on a same side of the strip-shaped body 100, and the flexible elastic plate 300 is clamped between the strip-shaped body 100 and the inflatable bladder 200, and the flexible elastic plate 300 is made of a metal including but not limited to chromium steel or chromium-vanadium steel. The flexible elastic plate 300 has a curling elasticity in a fixed direction, such that the flexible elastic plate will remain in a rigid straight status if the flexible elastic plate 300 is pulled straight. If an external force is exerted onto the flexible elastic plate 300 to bend the flexible elastic plate 300, the flexible elastic plate 300 will produce an elastic restoring force immediately, so that the flexible elastic plate 300 will be curled and deformed into a spiral form again.

In addition, the present invention is not limited to a single flexible elastic plate only, but a combination of flexible elastic plates can be used. For example, two or three flexible elastic plates, one long and one short flexible elastic plates, one wide and one narrow flexible elastic plates, or two flexible elastic plates made of different materials may be adopted in the present invention without departing from the scope and spirit of the invention set forth in the claims.

The periphery of the bottom lining 400 is coupled to the strip-shaped body 100, and the bottom lining 400 and the strip-shaped body 100 are used for jointly covering the inflatable bladder 200 and the flexible elastic plate 300, so that it is not necessary to sew or fix the inflatable bladder 200 to the strip-shaped body 100 directly, and the bottom lining 400 is made of a soft material such as a flannel fabric with a better texture.

The male adhesive layer 500 is attached onto the exterior of the bottom lining 400, and the female adhesive layer 600 is attached onto another side of the strip-shaped body 100. In other words, the male adhesive layer 500 and the female adhesive layer 600 are disposed on different sides of the strip-shaped body 100, and the male adhesive layer 500 and the female adhesive layer 600 are attached to each other, and the male adhesive layer 500 is a VELCRO® tape, and the female adhesive layer 600 is a piece of flannel fabric, and the female adhesive layer 600 includes a through hole 610 interconnected to the interconnecting hole 110.

The vent connector 700 is installed onto the inflatable bladder 200 and passed through the inflation hole 220, the interconnecting hole 110, and the through hole 610, such that air can be passed through the vent connector 700 and entered into the air chamber 210. In practical applications, the vent connector 700 is coupled to the inflatable bladder 200 by a hot pressing method, and a major portion of the vent connector 700 is situated in the air chamber 210, and only a small portion is exposed from the inflatable bladder 200.

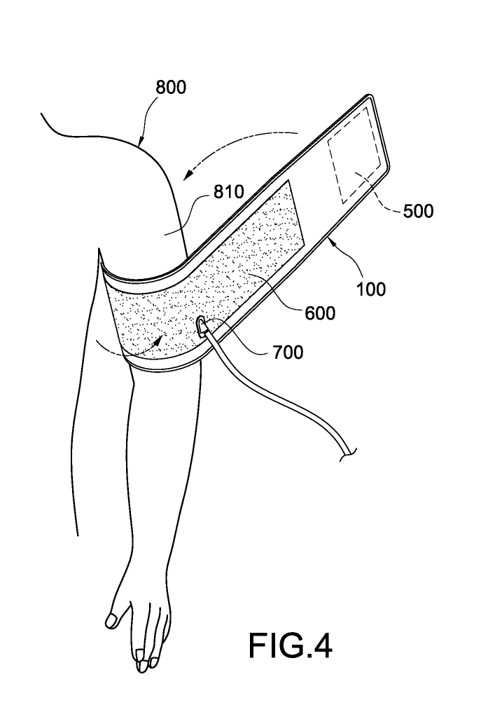

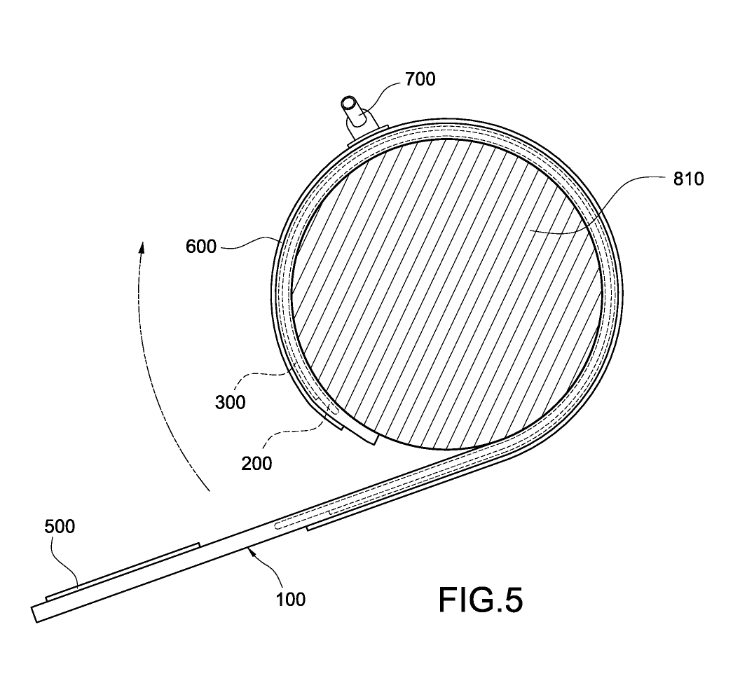

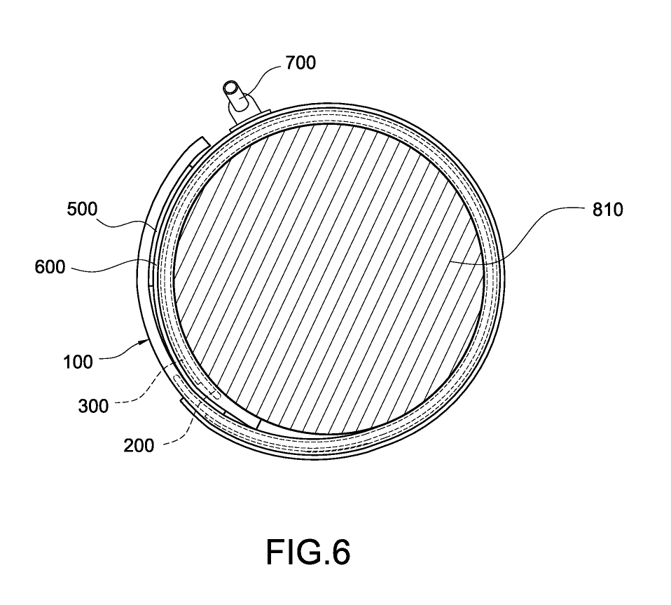

In an application of the present invention as shown in FIGS. 4 to 6, the flexible elastic plate 300 is substantially in a rigid straight status at the beginning, and an end of the bottom lining 400 without the male adhesive layer 500 is pressed at a testing position 810 of a human body 800, wherein the testing position 810 is an arm, and the female adhesive layer 600 faces outward, and an external force is applied to the flexible elastic plate 300 to bend the flexible elastic plate 30 slightly inward, such that the flexible elastic plate 300 will be curled and deformed. Now, external forces are no longer needed, and the flexible elastic plate 300 can be curled and contracted automatically by the elastically restoring force of the flexible elastic plate 300, so as to drive the strip-shaped body 100 to wind at the testing position 810, and the male adhesive layer 500 and the female adhesive layer 600 are attached to each other to achieve the positioning effect.

The flexible elastic plate 300 wound and compressed at the testing position 810 provides a basic compression force to the testing position 810 to prevent errors or a low measuring accuracy of the conventional method, and the invention also provides an easy and convenient operation and a quick way of positioning the cuff. The invention comes with a very simple structure and a low cost to enhance the product competitiveness.

The inflatable bladder 200 is wound at the testing position 810 through the bottom lining 400. Now, the inflatable bladder 200 can be inflated to compress the testing position 810, and then blood pressures can be measured. Since the bottom lining 400 is made of a soft material, so that the accuracy of blood pressure measurements will not be affected. The testing position 810 is simply in contact with the bottom lining 400, and users will not feel uncomfortable due to any sharp pain.

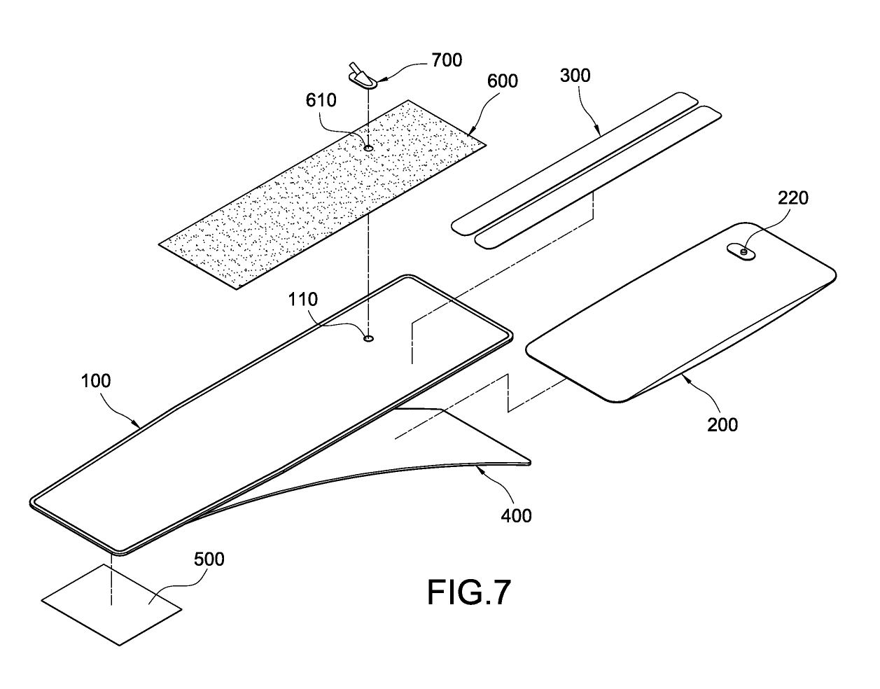

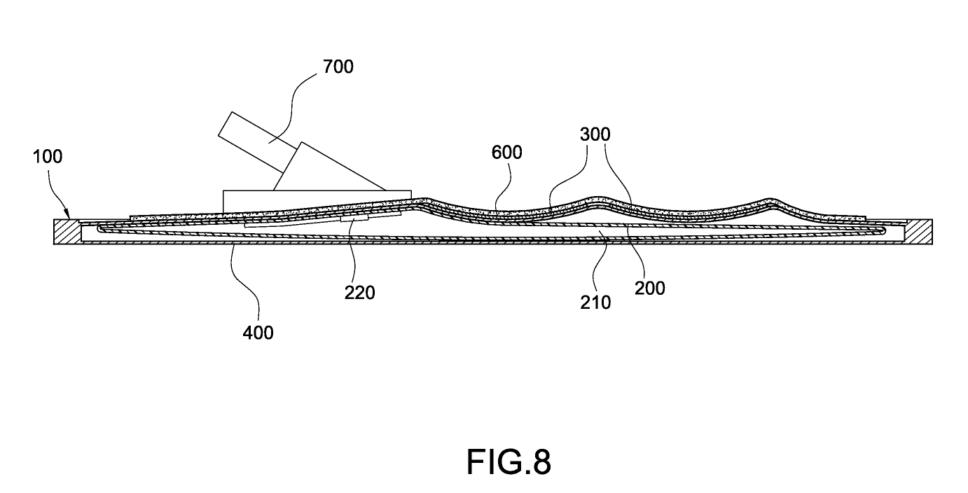

With reference to FIGS. 7 and 8, the flexible elastic plate 300 is attached onto the strip-shaped body 100 and along the lengthwise direction of the strip-shaped body 100. The flexible elastic plate 300 and the inflatable bladder 200 are installed on different sides of the strip-shaped body 100, and the flexible elastic plate 300 is clamped between the strip-shaped body 100 and the female adhesive layer 600, such that the flexible elastic plate 300 will not be in a direct contact with the inflatable bladder 200, since the flexible elastic plate 300 is made of a harder material. As a result, damages to the soft the inflatable bladder 200 by the flexible elastic plate 300 can be avoided.

In addition, the female adhesive layer 600 and the strip-shaped body 100 are integrally formed. In other words, the strip-shaped body 100 is a piece of flannel fabric that can be attached directly to the male adhesive layer 500.

While the invention has been described by means of specific embodiments, numerous modifications and variations could be made thereto by those skilled in the art without departing from the scope and spirit of the invention set forth in the claims.

What is claimed is:

1. A coiling blood pressure cuff, provided for winding and compressing a testing position of a human body, and the coiling blood pressure cuff comprising:
a strip-shaped body;
an inflatable bladder, installed at the strip-shaped body; and
a flexible elastic plate, attached onto the strip-shaped body and along the lengthwise direction of the strip-shaped body;
thereby, if an external force is exerted onto the flexible elastic plate to curl and deform the flexible elastic plate, the flexible elastic plate will be curled and contracted elastically and automatically to drive the strip-shaped body to be wound and positioned at the testing position.

2. The coiling blood pressure cuff of claim 1, wherein the flexible elastic plate and the inflatable bladder are installed on same side of the strip-shaped body.

3. The coiling blood pressure cuff of claim 1, wherein the flexible elastic plate and the inflatable bladder are installed on different sides of the strip-shaped body.

4. The coiling blood pressure cuff of claim 1, wherein the flexible elastic plate is made of chromium steel.

5. The coiling blood pressure cuff of claim 1, wherein the flexible elastic plate is made of chromium-vanadium steel.

6. The coiling blood pressure cuff of claim 1, further comprising a bottom lining coupled to the strip-shaped body, and the bottom lining and the strip-shaped body are jointly covered onto the inflatable bladder.

7. The coiling blood pressure cuff of claim 6, further comprising a male adhesive layer disposed on an external side of the bottom lining and a female adhesive layer disposed on an external side of the strip-shaped body, and the male adhesive layer and the female adhesive layer are attached with each other.

8. The coiling blood pressure cuff of claim 7, wherein the inflatable bladder includes an air chamber therein, and an inflation hole formed on the inflatable bladder and interconnected to the air chamber.

9. The coiling blood pressure cuff of claim 8, wherein the strip-shaped body includes an interconnecting hole interconnected to the inflation hole, and the female adhesive layer includes a through hole interconnected to the inflation hole.

10. The coiling blood pressure cuff of claim 9, further comprising a vent connector installed to the strip-shaped body and connected to the inflation hole, the interconnecting hole, and the through hole.
